(19) United States
(12) Patent Application Publication
(10) Pub. No.: US 2026/0241109 A1
(43) Pub. Date: Aug. 20, 2026

(54) AUTONOMOUS DOSE INJECTION

(71) Applicant: BIGFOOT BIOMEDICAL, INC, Milpitas, CA (US)

(72) Inventor: Matthew CLEMENTE, San Jose, CA (US)

(21) Appl. No.: 18/871,377

(22) PCT Filed: Jun. 2, 2023
(86) PCT No.: PCT/US2023/067867

§ 371 Date: Dec. 3, 2024

Related U.S. Application Data
(60) Provisional application No. 63/365,849, filed on Jun. 3, 2022.
(60) Provisional application No. 63/382,443, filed on Nov. 4, 2022.

(51) Int. Cl. A61M 5/315; A61M 5/20; A61M 5/24

(52) U.S. Cl. CPC A61M 5/31546; A61M 5/2422; A61M 5/31568; A61M 2005/2093; A61M 2205/3303; A61M 2205/3592; A61M 2205/502; A61M 2205/52; A61M 2230/201

(57) ABSTRACT

Systems, methods, and devices relate, generally, to autonomous dose injection. One or more examples relate to a durable insulin injection device. Such a durable dose injection device may include a chamber, a drive mechanism, a logic circuit, and an actuator. The chamber may be to receive a prefilled insulin cartridge. The drive mechanism may be to apply force to a plunger of a received prefilled insulin cartridge, the plunger arranged to move within the received cartridge responsive to force applied by the drive mechanism and thereby push fluid in the received cartridge toward an interface for dispensing fluid. The logic circuit may be to receive analyte data for the user and calculate a target amount of insulin to deliver to a user. The dose-ready confirmation actuator may be for the user to actuate to cause the delivery of insulin to the user. In one or more examples, the durable insulin injection device does not provide the user with an indication of the calculated target amount of insulin for non-zero calculated target amounts of insulin.

### CROSS-REFERENCE TO RELATED APPLICATIONS

This application claims priority to U.S. Application Serial Nos. 63/365,849, filed on Jun. 3, 2023 and 63/382,443, filed on Nov. 4, 2022. The disclosures of the prior applications are considered part of the disclosure of this application, and are incorporated in their entirety into this application.

### FIELD

One or more examples relate, generally, to injecting doses of a therapeutic agent, including medicine and insulin, and devices and systems for the same.

### BACKGROUND

When healing is not available for a medical condition, treatments and therapies are sometimes used to reduce or control the medical condition. For example, one or more therapeutic agents may be administered to the body to reduce or control a medical condition or its symptoms. As a non-limiting example, a biologically effective substance (a “drug”) may be injected, infused, or inhaled into the body to reduce, prevent or otherwise control a medical condition or its symptoms. Sometimes multiple administrations of one or more drugs are required a day, and so a patient or the patient's medical provider or caregiver will plan, in advance, the times, types of drugs, and amounts of the drugs to administer. If a medical conditioned is self-managed, a patient may have to self-monitor their physiological state or biological characteristics via biological samples (e.g., blood or tissue samples, without limitation) or other means and self-administer a drug. To self-administer, the patient may have to determine the amount of a drug to administer, which could be based on a variety of physiological or lifestyle factors. Accordingly, a patient faces a considerable cognitive burden to self-manage their medical condition.

### BRIEF DESCRIPTION OF THE DRAWINGS

To easily identify the discussion of any particular element or act, the most significant digit or digits in a reference number refer to the figure number in which that element is first introduced.

FIG. 1 illustrates an aspect of the subject matter in accordance with one or more examples.

FIG. 2A illustrates an aspect of the subject matter in accordance with one or more examples.

FIG. 2B illustrates an aspect of the subject matter in accordance with one or more examples.

FIG. 2C illustrates an aspect of the subject matter in accordance with one or more examples.

FIG. 2D illustrates an aspect of the subject matter in accordance with one or more examples.

FIG. 3 illustrates an aspect of the subject matter in accordance with one or more examples.

FIG. 4 illustrates an aspect of the subject matter in accordance with one or more examples.

FIG. 5 illustrates an aspect of the subject matter in accordance with one or more examples.

FIG. 6 illustrates an aspect of the subject matter in accordance with one or more examples.

FIG. 7 illustrates an aspect of the subject matter in accordance with one or more examples.

FIG. 8 illustrates an aspect of the subject matter in accordance with one or more examples.

FIG. 9 illustrates an aspect of the subject matter in accordance with one or more examples.

FIG. 10 illustrates an aspect of the subject matter in accordance with one or more examples.

FIG. 11 illustrates an aspect of the subject matter in accordance with one or more examples.

FIG. 12 illustrates an aspect of the subject matter in accordance with one or more examples.

FIG. 13 illustrates an aspect of the subject matter in accordance with one or more examples.

FIG. 14 illustrates an aspect of the subject matter in accordance with one or more examples.

FIG. 15 illustrates an aspect of the subject matter in accordance with one or more examples.

FIG. 16 illustrates an aspect of the subject matter in accordance with one or more examples.

FIGS. 17A-17F illustrate various perspective views of a dose injection device in accordance with one or more examples.

### DETAILED DESCRIPTION

In the following detailed description, reference is made to the accompanying drawings, which form a part hereof, and in which are shown, by way of illustration, specific examples of examples in which the present disclosure may be practiced. These examples are described in sufficient detail to enable a person of ordinary skill in the art to practice the present disclosure. However, other examples enabled herein may be utilized, and structural, material, and process changes may be made without departing from the scope of the disclosure.

The illustrations presented herein are not meant to be actual views of any particular method, system, device, or structure, but are merely idealized representations that are employed to describe the examples of the present disclosure. In some instances, similar structures or components in the various drawings may retain the same or similar numbering for the convenience of the reader; however, the similarity in numbering does not necessarily mean that the structures or components are identical in size, composition, configuration, or any other property.

The following description may include examples to help enable one of ordinary skill in the art to practice the disclosed examples. The use of the terms “exemplary,” “by example,” and “for example,” means that the related description is explanatory, and though the scope of the disclosure is intended to encompass the examples and legal equivalents, the use of such terms is not intended to limit the scope of an example nor this disclosure to the specified components, steps, features, functions, or the like.

It will be readily understood that the components of the examples as generally described herein and illustrated in the drawings could be arranged and designed in a wide variety of different configurations. Thus, the following description of various examples is not intended to limit the scope of the present disclosure, but is merely representative of various examples. While the various aspects of the examples may be presented in the drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

Those of ordinary skill in the art would understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative logical blocks, modules, and circuits described in connection with the examples disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to examples of the present disclosure.

The examples may be described in terms of a process that is described or depicted as a flow process, flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

Any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may comprise one or more elements.

As used herein, the term “substantially” in reference to a given parameter, property, or condition means and includes to a degree that one of ordinary skill in the art would understand that the given parameter, property, or condition is met with a small degree of variance, such as, for example, within acceptable manufacturing tolerances. By way of example, depending on the particular parameter, property, or condition that is substantially met, the parameter, property, or condition may be at least 90% met, at least 95% met, or even at least 99% met.

In this description the term “coupled” and derivatives thereof may be used to indicate that two elements co-operate or interact with each other. When an element is described as being “coupled” to another element, then the elements may be in direct physical or electrical contact or there may be intervening elements or layers present. In contrast, when an element is described as being “directly coupled” to another element, then there are no intervening elements or layers present. The term “connected” may be used in this description interchangeably with the term “coupled,” and has the same meaning unless expressly indicated otherwise or the context would indicate otherwise to a person having ordinary skill in the art.

Diabetes mellitus is a chronic metabolic disorder caused by the inability of a person's pancreas to produce sufficient amounts of the hormone insulin such that the person's metabolism is unable to provide for the proper absorption of sugar and starch. This can lead to hyperglycemia or hypoglycemia. Hyperglycemia is the presence of an excessive amount of glucose within the blood plasma. Persistent hyperglycemia has been associated with a variety of serious symptoms and life-threatening long-term complications such as dehydration, ketoacidosis, diabetic coma, cardiovascular diseases, chronic renal failure, retinal damage, and nerve damages with the risk of amputation of extremities. Hypoglycemia is where the amount of glucose within the blood plasma falls below a standard range. Hypoglycemia has been associated with a variety of symptoms such as clumsiness, trouble talking, confusion and life-threatening long-term complications such as loss of consciousness, seizures, or death. Because healing is not yet possible, a permanent therapy is necessary which provides constant glycemic control to constantly maintain the level of blood glucose within normal limits. Such glycemic control is achieved by regularly supplying external drugs to the body of the patient to thereby reduce the elevated levels of blood glucose.

An external biologically effective drug (e.g., insulin or its analog) is commonly administered by means of daily injections, in some cases, multiple daily injections (MDI) of a rapid-acting insulin or long-acting insulin via reusable transdermal liquid dosing devices (commonly referred to as an “insulin pen”) or hypodermic syringes. The injections are typically administered by a person with diabetes (PWD), and so require self-monitoring of blood glucose and the self-administration of insulin. The PWD that manages their care using MDI often plans insulin injections for each day, in advance, based on basal insulin requirement as well as external factors such as meals, exercise, sleep, without limitation. A typical dosing plan will include the time of day each dose, the type of insulin(s) (e.g., fast acting, long acting, or a mixture of fast acting and long acting, without limitation) for each dose, and amount of insulin for each dose. In addition, PWDs will self-monitor their blood glucose and self-administer “bolus” dose(s) of rapid-acting insulin if their blood glucose is too high or consume carbohydrates (or sometimes administer glycogen) if their blood glucose is too low.

The “correct” insulin dose is a function of the level of glucose in the blood, physiological factors such as a person's insulin sensitivity, and lifestyle factors such as meals (e.g., recently consumed carbohydrates that have yet to be metabolized into glucose and absorbed into the blood). Even with careful planning and self-monitoring, a PWD may skip doses, double dose, and dose the wrong amount and/or type of insulin. Insufficient insulin can result in hyperglycemia, and too much insulin can result in hypoglycemia.

Accordingly, PWDs face a considerable cognitive burden to self-monitor their blood glucose and self-administer their insulin.

A variety of diabetes treatment devices have been developed to assist with self-monitoring (e.g., blood glucose meters (BGM), continuous glucose monitors (CGM), therapy applications, without limitation) and to assist with self-administration of insulin (e.g., injection pens, infusion pumps, and bolus calculators, without limitation).

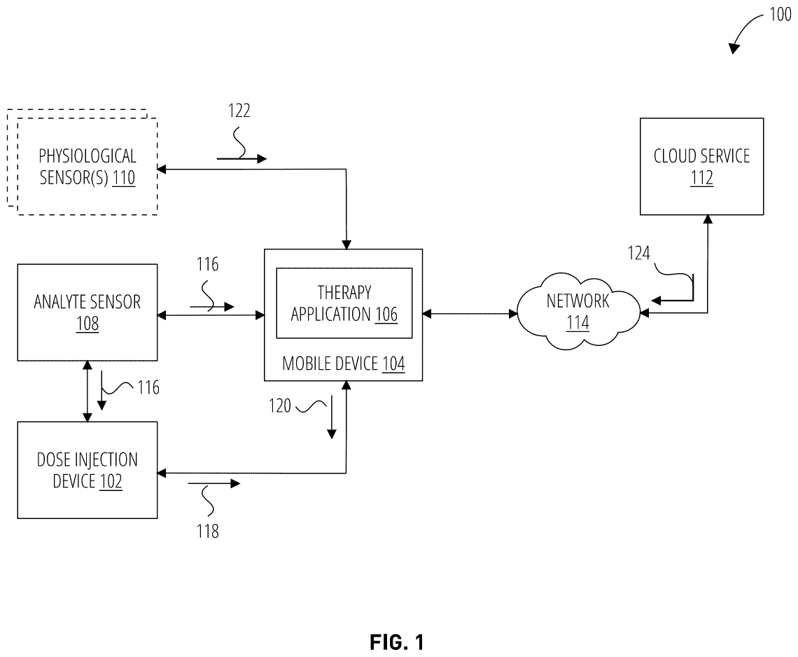

FIG. 1 is a block diagram depicting a system 100 to assist with drug therapy management (also referred to herein as a “therapy management system 100”) and diabetes management more generally, in accordance with one or more examples. As depicted in FIG. 1, system 100 may include a dose injection device 102, a mobile device 104, a therapy application 106, a analyte sensor 108, a physiological sensor(s) 110, a cloud services 112, and a network 114.

Dose injection devices 102 is configured for autonomous dose injection as discussed herein and may also be referred to herein as an “autonomous dose injection device 102.” Here, when a dose injection is described as “autonomous,” it means that the dose injection device 102 controls the calculation and delivery of dose amounts associated with an autonomous dose injection without informing a user of values of a calculated dose amount or of a delivered dose amount. The dose injection device 102 makes the user “dosing aware” (i.e., provides notification that a dose was dispensed) but does not make the user “calculated dose amount” aware or “delivered dose amount” aware. A user is therefore unaware of the calculated dose amounts or delivered dose amounts at least via dose injection device 102, though may learn these amounts via other sources as a non-limiting example, via therapy application 106 or cloud service 112. In one or more examples, dose injection device 102 may display calculated analyte values or indications of whether a user's analyte level is within predetermined ranges (e.g., within a normal range unassociated with a risk, such as a risk of hyperglycemia or hypoglycemia, within a range associated with a risk, such as within a range associated with hyperglycemia, or within a range associated with hypoglycemia, without limitation). In this manner, the cognitive burden on a user of dose injection device 102 may be reduced to monitoring whether or not their analyte level, and more generally, their medical condition, is being managed.

As a non-limiting example, in one or more examples, 102 may display calculated glucose values or indications of whether a user's blood glucose is within predetermined ranges (e.g., within a normal range unassociated with risk of hyperglycemia or hypoglycemia, within a range associated with hyperglycemia, or within a range associated with hypoglycemia, without limitation). In this manner, the cognitive burden on a user of dose injection device 102 may be reduced to monitoring whether or not their blood glucose, and more generally, their diabetes, is being managed.

Dose injection device 102 is configured to deliver doses of a drug to the body. In one or more examples, dose injection device 102 delivers the drug via an injector such as a needle, without limitation. In one or more examples, dose injection device 102 is an injector pen configured to deliver a drug into the subcutaneous tissue of the body of a person. In one or more examples, dose injection device 102 may be a durable injector pen configured to receive a pre-filled, disposable, drug cartridge.

In one or more examples, the drug may be insulin, an analog of insulin, or a similar therapeutic agent, and dose injection device 102 may be configured to receive a pre-filled, disposable, insulin cartridge.

Mobile device 104 is a mobile computing device to receive, send, store, process, and/or display data as well as execute application software. Non-limiting examples of mobile computing devices include a smart phone, a personal data assistant (PDA), a tablet computer, a laptop computer, or a wearable computer (e.g., a smart watch or smart eyeglasses, without limitation). In one or more examples, mobile device 104 includes, without limitation: a memory and at least one processor to execute application software stored on the memory (e.g., therapy application 106, without limitation), and communication equipment to wirelessly communicate with one or more of analyte sensor 108, dose injection device 102, cloud service 112 or optional physiological sensor(s) 110.

Therapy application 106 is application software configured, generally, to perform various features and functions of therapy management discussed herein. Therapy application 106 may connect to, and communicate with, other components of system 100 including analyte sensor 108, dose injection device 102, cloud service 112 and physiological sensor(s) 110. Therapy application 106 may act as an interface between a user (e.g., a PWD, caregiver, healthcare provider, without limitation) and an drug therapy of a PWD. In one or more examples, therapy application 106 may receive, aggregate, process, and present therapy data of a PWD and present the therapy data to a user of therapy application 106 via, e.g., a display (not depicted) of mobile device 104. Therapy data that therapy applications 106 receive, aggregate, process and present may include one or more of: analyte data 116 received from analyte sensor 108 (as discussed below), dosing data 118 received from dose injection device 102 (as discussed below), physiological data 122 received from physiological sensor(s) 110 (as discussed below), or therapy parameters 124 received from cloud service 112.

Analyte sensor 108 is a device configured to generate analyte data, including values that represent analyte present in a person's bloodstream or body, more generally. Such presence of an analyte in a person's body is referred to herein as “body analyte,” and values that represent body analyte at least partially based on samples taken from the body are referred to herein as “analyte values.”

As a non-limiting example, 108 may be considered as a glucose sensor denoted glucose sensor 108′. The presence of glucose in a person's bloodstream referred to herein as “blood glucose.” In one or more examples, glucose sensor 108′ may be configured to use any suitable indication of glucose present in a person's bloodstream, such as glucose present in a person's veins or arteries, to estimate blood glucose. As a non-limiting example, glucose sensor 108′ may be configured to sample interstitial fluid glucose levels of a user's body, determine values that represent blood glucose at least partially based on the samples, and store glucose data that includes, or is at least partially based on, the estimated glucose values (also referred to herein as “glucose values”). In various examples, the glucose data generated by glucose sensor 108 may further include information about estimated glucose values such as date and time that estimated glucose values were determined. Glucose sensor 108′ may be, as non-limiting examples, a continuous glucose monitor (CGM), a flash glucose monitor (FGM), a blood glucose meter (BGM), or any other suitable sensor for determining values representative of blood glucose levels in a body at least partially based on interstitial fluid glucose levels or intermediate measurements thereof.

In one or more examples, analyte sensor 108 may include equipment to communicate analyte data 116 generated by analyte sensor 108, for example, to therapy application 106 or dose injection device 102, without limitation. As a non-limiting example, analyte data 116 at analyte sensor 108 may be communicated via Near Field Communication (NFC), for example, via an NFC tag at analyte sensor 108 and an NFC reader at mobile device 104 or dose injection device 102.

A physiological sensor of optional physiological sensor(s) 110 include any sensor suitable to capture information about the physiological state of a person and provide the information as physiological data 122 to mobile device 104. As non-limiting examples, physiological sensor(s) 110 may include a heart rate monitor, thermometer, or electrocardiogram, without limitation. Physiological sensor(s) 110 may be individual devices capable of communication with therapy application 106 or sensors in a multi-use device for capturing multiple types of physiological data 122. In or more examples, one or more physiological sensor(s) 110 may include in a smart device for which capturing physiological data 122 is one of several services performed by the smart device, such as smart watch.

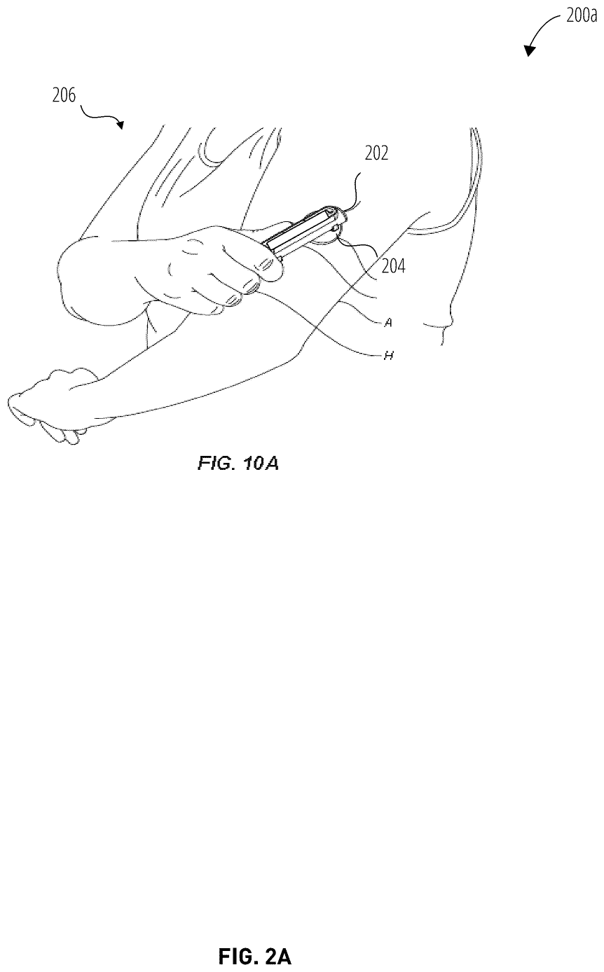

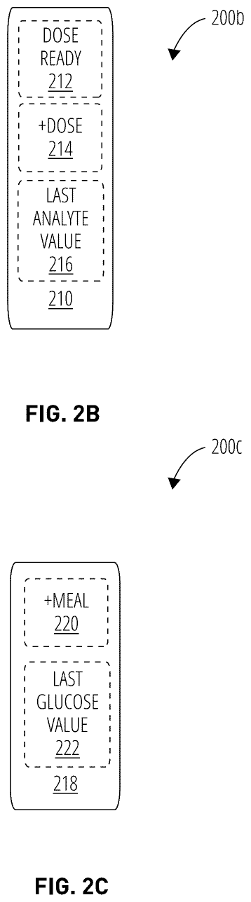

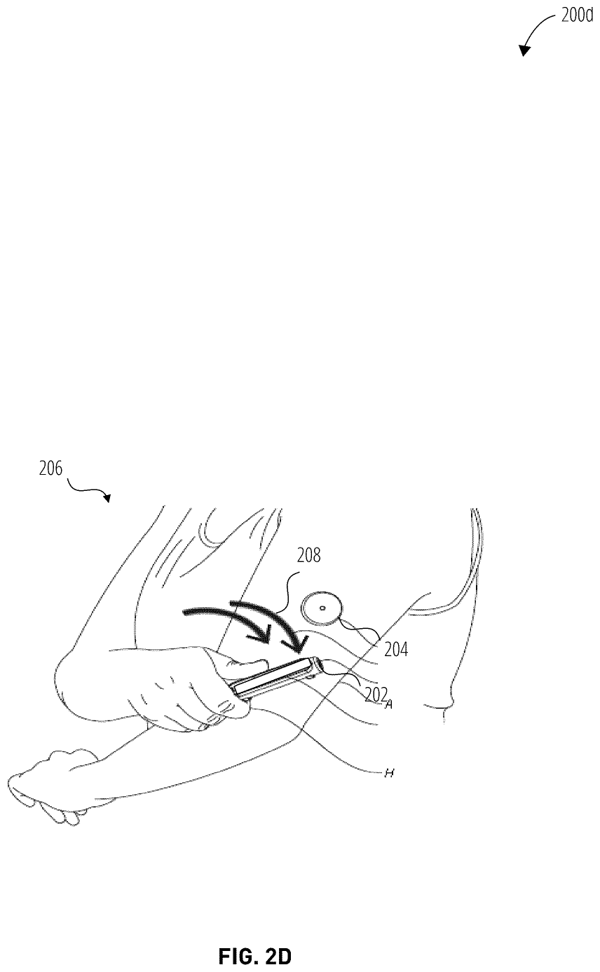

FIG. 2A and FIG. 2D depict an example of a system 100, hereinafter systems 200a and 200d, and a contemplated use of system 200a and system 200d by a user 206 in accordance with one more examples.

FIG. 2A depicts an example of dose injection device 102, hereinafter dose injection device 202, in a hand H of user 206 being swiped over an example of a analyte sensor 108, hereinafter analyte sensor 204, to receive analyte data (e.g., analyte data 116, without limitation) from analyte sensor 204 via NFC. In one or more examples, analyte data may be received via alternative channels (e.g., Bluetooth, Bluetooth Low Energy (BLE), without limitation) or received via an example of a mobile device 104 (example mobile device not depicted by FIG. 2A).

In one or more examples, dose injection device 202 is configured to display information about a user's body analyte, for example, to display one or more of: an analyte value, an indication of whether, or not, a user's analyte value is within a desired range of analyte values, an indication that a dose is ready, or an indicate that a dose was delivered.

As a non-limiting example, dose injection device 202 may be configured to display information about a user's blood glucose, for example, to display one or more of: an glucose value, an indication of whether, or not, a user's glucose value is within a desired range of glucose values (e.g., normal, out-of-range—hypoglycemic, out-of-range—hyperglycemic), an indication that a dose is ready, or an indicate that a dose was delivered.

FIG. 2B and FIG. 2C are diagrams depicting example display screens, here display screen 200b and display screen 200c, for a dose injection device 102 such as dose injection device 202, without limitation, in accordance with one or more examples.

In one or more examples, display screens 200b and 200c may be configured for touch or pressure sensing, as a non-limiting example, incorporating capacitive or resistive sensing systems, which facilitate user interaction with a user interface that enables a user to interact with dose injection device 102 and system 100 more generally. In one or more examples, portions of display screens 200b and 200c or apparatuses coupled to display screens 200b and 200c may include buttons, dials, or switches that provide some or a totality of the functions of user interfaces 210 and 218.

In one or more examples, display screen 200b may display a user interface 210 to enable a user to interact with dose injection device 102. User interface 210 may include dose ready notification 212, dose ready confirmation 214, and dose ready confirmation 214.

In one or more examples, dose ready notifications 212 is configured to notify a user that a dose of medication is ready to be delivered. In one or more examples, dose ready notification 212 may be a visual indicator (e.g., blinking light, icon, display text, (e.g., “DOSE READY,” without limitation)) and may optionally be accompanied by an audio indicator (e.g., a pre-determined tone or tones audible to a person, without limitation). In one or more examples, dose ready notification 212 does not include a value of a target dose amount nor any other information about a target dose amount. This reduces confusion for a user, as most users merely need to be aware that a dose is ready to be injected and presume that a target dose amount has been calculated that will appropriately control their analyte levels. As a non-limiting example, a typical PWD will presume that a target dose amount of insulin has been calculated that will appropriate control their glucose levels.

In one or more examples, last analyte value information 216 is configured to provide information to a user about a last (e.g., recent, without limitation) analyte value that represents the user's blood glucose. Such an analyte value may have been included in, or at least partially based on, analyte data 116 received from a analyte sensor, as a non-limiting example, at least partially in response to dose injection device 202 being swiped by a analyte sensor 204 as depicted by FIG. 2A.

As a non-limiting example, a last analyte value information 216 may include a glucose value included in, or at least partially based on, glucose data received from a glucose sensor, as a non-limiting example, at least partially in response to dose injection device being swiped by a glucose sensor. Such a glucose value may be in milligrams per deciliter (mg/dL).

In some cases, a user may be distracted or confused if a dose injection device displays an analyte value. For example, if a user presumes a dose injection device will calculate a proper dose of insulin and notify the user that the dose is ready at a proper time, the purpose of a specific analyte value may confuse the user, and they may delay their therapy. In some cases, an indication that a recent analyte value has been received may suffice for a user so that a user can initiate actions to read analyte data 116 from analyte sensor 108 if a recent value has not been received.

In one or more examples, last analyte value information 216 may not present an actual value of blood glucose, and may be an indication that a analyte value has been received within a threshold period of time that it is presumably safe for a user to dose a drug, or that a analyte value has not been received within threshold period of time so it may not be safe for a user to dose a drug and a recent analyte value should be updated before dosing a drug.

In one or more examples, dose ready confirmation 214 is configured to enable a user to confirm to the dose injection device 102, that the user is ready to deliver a dose of a drug. The user is unaware of the target dose amount calculated at dose injection device 102, so the dose ready confirmation 214 does not confirm or validate the calculated target dose amount associated with a dose ready notification 212.

Upon receipt of dose ready confirmation 214, a dose injection device 102 can then initiate injection of insulin, optionally if all other conditions are met.

In one or more examples, dose injection device 102 controls user interface 210 to generate the dose ready notification 212 at least partially in response to determination of a target dose amount. In one or more examples, the target dose amount utilized by the dose injection device 102 to deliver a dose of a drug may be different than the target dose amount utilized to generate the dose ready notification 212. As a non-limiting example, dose injection device 102 may change a value of a target dose amount in response to various conditions such as receipt of a new analyte value indicative of a different target dose amount of a drug. Notably, a user may be unaware of the change because a value of a target dose amount is not provided to the user.

As a non-limiting example, the target dose amount may be for a target dose amount of insulin. The dose injection device 102 may be configured to change a value of a target dose amount of insulin in response to a condition such as receipt of a meal announcement after the notification was generated but before a dose of insulin was injected, or receipt of a new glucose value indicative of a different target dose amount of insulin.

FIG. 2C is a diagram depicting an example display screen 200c for a dose injection device 102, in accordance with one or more examples. An example display screen of a dose injection device 102 may be configured as display screen 200B, display screen 200c, or to alternately display screen 200b or display screen 200c.

In one or more examples, display screen 200c may display user interface 218 to enable a user to interact with a dose injection device 102. User interface 218 includes last glucose value information 222 and meal announcement 220. Last glucose value information 222 may be configured similar to last glucose value information 216.

In one or more examples, meal announcements 220 is configured to enable a user to provide an indication that the user recently consumed a meal. Meal announcement 220 may optionally include meal announcement categorization, such as a size categorization, e.g., “small,” “medium,” or “large,” without limitation. Amounts of carbohydrates associated with meal size may be pre-set, and the amounts may be personalized, as a non-limiting example, at least partially based on values provided by therapy application 106 or cloud service 112. Personalized values may be at least partially based on a user's average meal sizes. As a non-limiting example, therapy application 106 may include or communicate with a meal tracking application and infer meal sizes for small, medium, and large meals based on meal data input to the meal tracking application. In another example, a user may enter values for amount of carbohydrates associated with small, medium, or large meals utilizing therapy application 106.

In one or more examples, meal announcement 220 may be associated with a number for carbohydrates that is personalized for a user based on other user-specific dosage parameters entered by the user via therapy application 106 for system 100 (e.g., total daily long-acting insulin dosage (e.g., U/day), a total daily dose of insulin (e.g., total of long and quick acting), a carbohydrate-to-insulin ratio, an insulin sensitivity factor, a glucose setpoint, or a combination thereof). In some cases, the value of carbohydrates assigned to meal announcement 220 and respective categories of meals included therewith may be personalized over time based on estimations of the size of each meal consumed when a respective meal announcement 220 button is selected based on a glucose response after the consumption of each meal. In some cases, the number of carbohydrates assigned to each preset icon or button may be optionally rounded to the nearest 5 grams of carbohydrates and displayed. In one or more examples, a number of carbohydrates for each button is not displayed. In one or more examples, a number of carbohydrates assigned to each user-selectable icon or button can be initially set at a predetermined starting point or can be determined based on entered user information, and then iteratively adjusted upward or downward based upon the glycemic response to that selected meal size and bolus over time.

In one or more examples, initial settings for meal announcement 220 may be preset with predetermined values or ranges (e.g., small=20 g or 15-25 g, medium=30 g or 30-45 g, and large=50 g or 50-75 g). Additionally, or alternatively, the initial settings may be set based on entered user data or based on one or more user-specific dosage parameters entered via therapy application 106. In some cases, initial settings for the one or more user-selectable icons or buttons can be based on an initially entered or determined and programmed total daily long-acting insulin (TDLAI) dose (e.g., U/day). For example, the relationship between the LAI dose [U/day] and Geometric Mean Meal Size [g] as characterized by the line corresponding to the major axis of the hyper ellipsoid is: μ*MS=12.1*BR0.387. The relationship between Geometric Mean Meal Size [g] and Geometric Standard Deviation Meal Size is: σ*MS=1.92−μ*MS/186 where MS may represent the meal size and BR may represent the basal rate of insulin.

Accordingly, initial meal size groups may correspond to predetermined percentiles of the Meal Size distribution by combining the above equations, optionally rounding meal size groups to the nearest 1, 5, or 10 grams. In some cases, the relationship between typical meal sizes and other user-specific dosage parameters can be determined according to population statistics. In some cases, the number of carbohydrates associated with each user-selectable icon or button can be displayed on and/or adjacent to the user-selectable icon or button, which can help a user understand how to use the insulin delivery device or system to avoid deskilling the user. For example, seeing the number of carbohydrates assumed for each meal size helps a user that thinks about meals in terms of carbohydrates to adjust to using buttons to indicate a size of a meal. Additionally, by starting with display numbers rounded to the nearest 5 grams, the user can perceive that precision is not required, thus also reducing the cognitive burden on the user. Additionally, as the system iterates to personalize the amount of carbohydrates for each particular user-selectable icon or button, the system can adjust these numbers by smaller units (e.g., by 1 gram) to demonstrate to the user that the system is adjusting the number of carbohydrates associated with user-selectable icon or button.

Notably, neither display screen 200b nor display screen 200c includes information about an amount of a target dose to deliver or an actual dose delivered. In some cases, user may be confused by these values, as a non-limiting example, because the user presumes that a proper amount for a target dose is calculated and delivered.

Notably, a person having ordinary skill in the art would understand that display screen 200c may include inputs for announcements in addition to, or as alternatives to, meal announcement 220, as a non-limiting example, to announce physiological or lifestyle conditions that influence analyte levels in a persons body.

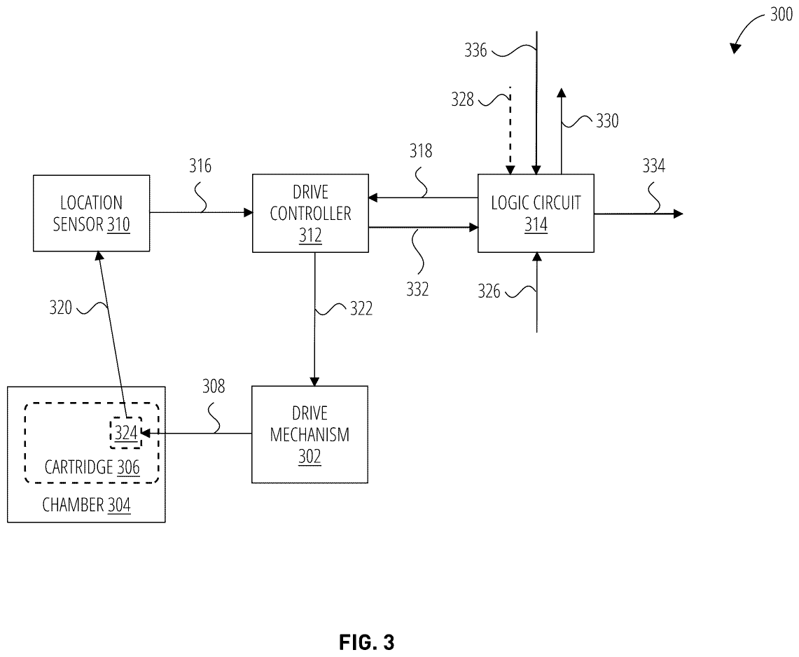

FIG. 2D depicts user 206 injecting 208 a drug via an example dose injection device 102, here dose injection device 202. Further information regarding various components of this dose injection device 202 are shown in FIG. 3.

FIG. 3 is a block diagram of an apparatus 300 to autonomously inject doses of a drug, in accordance with one or more examples. Apparatus 300 is a non-limiting example of dose injection device 102 of FIG. 1 or components thereof. As depicted by FIG. 3, apparatus 300 may include a drive mechanism 302, a chamber 304, a location sensor 310, a drive controller 312, and a logic circuit 324. Chamber 304 may be configured to hold an optional cartridge 306 having a plunger 324.

Logic circuit 314 is configured, generally, to calculate a target dose amount 318 to deliver, as non-limiting examples, to a user of apparatus 300 or to a user of dose injection device 102 more generally. In one or more examples, logic circuit 314 may calculate a value for target dose amount 318 at least partially responsive to therapy parameters 326 and optional meal or other lifestyle data 328. Therapy parameters 326 may include user-specific dosage parameters. In one or more examples, the user-specific dosage parameters may be for insulin therapy for treating diabetes, such as insulin sensitivity factor (ISF), carbohydrate ratio (CR), amount of daily dose of long-acting insulin (LAI), or a current BGL without limitation.

In one or more examples, values of at least some parameters of therapy parameters 326 may be updated at least partially based on values of therapy parameters 120 provided by therapy application 106. In one or more examples, values of at least some parameters of therapy parameters 326 may be updated by logic circuit 314.

Logic circuit 314 may be configured to provide a notification request 330 for a notification to indicate to a user that a dose is ready. Logic circuit 314 may provide the notification request 330 at least partially responsive to calculating a value for a target dose amount. Upon receiving a dosing confirmation 336, logic circuit 314 may send a request to deliver the target dose amount 318 to drive controller 312. In one or more examples, logic circuit 314 does not send a request to deliver target dose amount 318 unless it has received dosing confirmation 336 associated with target dose amount 318, and in one or more examples, does not send a request to deliver target dose amount 318 unless it receives dosing confirmation 336 within a predetermined period of time of calculating a value for target dose amount 318 or sending notification request 330.

Chamber 304 is a space or region at least partially defined in a housing of apparatus 300, or a dose injection device that includes apparatus 300 more generally, (housing not depicted), to receive and hold a cartridge 306 of a drug. In one or more examples, chamber 304 is configured to receive a prefilled cartridge 306, eject an exhausted cartridge 306 (e.g., an insufficient amount of a drug remains for a dose, without limitation), and optionally receive a prefilled cartridge 306 to replace (i.e., a “replacement cartridge 306”) an exhausted cartridge 306. In one or more examples, chamber 304 may be configured to non-removably hold a cartridge 306 until the cartridge 306 is exhausted. One or more walls of a body of cartridge 306 may surround a plunger 324 disposed within cartridge 306 to form a fluid reservoir of cartridge 306. A gasket of plunger 324 may be sized/dimensioned to create a tight (e.g., airtight or fluidically tight, without limitation) seal within cartridge 306 while still permitting plunger 324 to move within cartridge 306. In the case of a cartridge 306 that has not exhausted fluid therein, when plunger 324 moves it pushes fluid in the cartridge 306 toward an interface for dispensing fluid located at an opposite end of cartridge 306. Generally speaking, a volume of the fluid reservoir of cartridge 306 will be greater in a pre-filled state than the volume in an exhausted state.

Drive mechanism 302 is configured, generally, to apply a force 308 to a plunger 324 of a received cartridge 306, and via force 308 advance plunger 324 in cartridge 306 and dispense fluid therefrom. In one or more examples, drive mechanism 302 may include a piston that operatively couples to a plunger 324 of a cartridge 306 and via which force 308 is applied to plunger 324. In one or more examples, drive mechanism 302 may include an electric motor such as a DC or AC motor. In one or more examples, drive mechanism 302 may be a current controlled electric motor, a voltage controlled electric motor, or a pulse-width controlled electric motor. Drive mechanism 302 may be configured to generate force 308 at least partially responsive to control signals, such as control signals 322.

Drive controller 312 is configured, generally, to generate control signals 322 for drive mechanism 302. Control signals 322 may include first control signals to cause drive mechanism 302 to generate force 308 and/or apply force 308 to plunger 324, and second, different, control signals to cause drive mechanism 302 to not generate or stop generating force 308 and/or not apply or stop applying force 308 to plunger 324. Utilizing control signals 322, drive controller 312 may control a length of a period of time that drive mechanism 302 applies force 308 to plunger 324, and, indirectly, an amount of fluid dispensed from a cartridge 306.

Drive controller 312 may generate control signals 322 at least partially responsive to one or more of target dose amount 318 generated by logic circuit 314 or location signal 316 generated by location sensor 310. A distance that plunger 324 moves during a dosing operation generally corresponds to a displaced volume of fluid and, accordingly, a delivered amount of a drug. In one or more examples, drive controller 312 may monitor a location of plunger 324 via location signal 316 received from location sensor 310 as an indication of delivered amount of a drug.

In one or more examples, drive controller 312 may provide a signal to indicate an actual dose delivered (e.g., dose amount delivered 332, without limitation) to logic circuit 314. Notably, a dose amount delivered 332 during a dosing operation may differ from a target dose amount 318 requested by logic circuit 314, thus a value of dose amount delivered 332 provided by drive controller 312 may differ from a value of target dose amount 318 requested by logic circuit 314.

Differences between dose amount delivered 332 and target dose amount 318 may be due, as non-limiting examples, to non-linearities of or more of drive controller 312, drive mechanism 302, plunger 324 and cartridge 306, or location sensor 310. Logic circuit 314 may provide values for dose amount delivered 332 with dosing data 334/dosing data 118 to therapy application 106.

In one or more examples, drive controller 312 may provide dose amount delivered 332 at least partially responsive to detecting delivery of a dose. Drive controller 312 may include dose detection logic (not depicted) to detect delivery of a dose utilizing two or more locations of plunger 324 indicated by location signal 316. As a non-limiting example, drive controller 312 may store a first location of plunger 324 indicated by location signal 316 and compare it to a second location of plunger 324 indicated by location signal 316 at a later time.

Drive controller 312 may detect a dose at least partially responsive to determining that the first location and second location are different it.

Location sensor 310 may generate location signal 316 at least partially responsive to exhibited location 320 of plunger 324. In one or more examples, location sensor 310 may be any sensor suitable to reliability detect a location of plunger 324 and provide a lockout signal 510 indicative of the exhibited location 320.

In one or more examples, location signal 316 may be a sensor module that utilizes a light to interrogate plunger 324 and/or a cartridge 306. Such a sensor module includes one or more light sources (e.g., a light emitting diode (LED), without limitation) arranged to emit electromagnetic radiation, one or more light guides (e.g., a light tube, light pipe, light columniator, without limitation) to distribute the electromagnetic radiation across chamber 304, and one or more fixed detectors to optically couple to the one or more light guides/light sources via the distributed electromagnetic radiation. When an object made of an opaque material (a material that inhibits passage of light), such as the material of a plunger 324, is located in the path of the distributed light, the object disrupts the optical coupling of a detector with the light guide/light source. The output of a respective detector changes at least partially responsive to disrupted optical coupling. Because the locations of the detectors are fixed, a changed output of a respective detector may be correlated (e.g., via a lookup table, without limitation) by logic of drive controller 312 to a specific location of an object disrupting the optical coupling. In the case of a plunger 324 moving through a cartridge 306, when the plunger disrupts the optical coupling of a respective detector, the location of the plunger 324 may be deduced and provided via location signal 316.

In one or more examples, for a given dosing operation, drive controller 312 may know an initial or starting location of plunger 324. Upon instruction to deliver a target dose amount 318 by logic circuit 314, logic at drive controller 312 may determine an ending location of plunger 324 at least partially responsive to a target displaced volume that corresponds to the target dose amount 318, a starting location of plunger 324, and a total displaced volume per unit move of plunger 324. In one or more examples, an ending location may correspond substantially to a location of a respective detector of location sensor 310.

In one or more examples, an arrangement of detectors of location sensor 310 may be pre-selected such spacing between detectors corresponds to conventionally used dosing amounts for a drug, multiples of conventionally used dosing amounts for a drug, or fractions of conventionally used dosing amounts for a drug. As a non-limiting example, spacing between detectors may be chosen to correspond to 0.25 units of insulin, where a unit of insulin corresponds to one International Unit of insulin or 0.0347 milligrams of insulin.

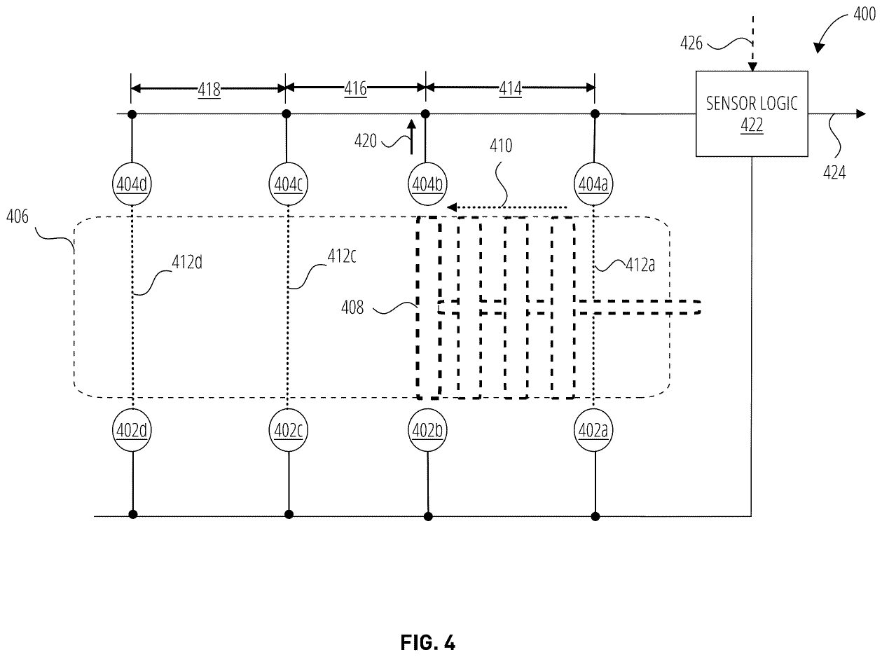

FIG. 4 is a diagram of an arrangement 400 of a location sensor (a location sensor arrangement 400), in accordance with one or more examples. As depicted by FIG. 4, location sensor arrangement 400 includes light sources 402a, 402b, 402c and 402d, arranged for optical coupling (optical coupling 412a, 412b, 412c and 412d) with detectors 404a, 404b, 404c, and 404d. Light guides are omitted for ease of illustration but may optionally be included.

Spacing 414, 416 and 418 respectively between detectors 404a, 404b, 404c and 404d may be substantially the same length, and generally correspond to a predetermined amount of insulin expected to be dispensed by advancing 410 plunger 408 within cartridge 406 for distances substantially equal to spacings 414, 416 and 418. In one or more examples, spacings may be chosen to correspond to fractions of Units of insulin (e.g., 0.1 Units, 0.2 Units, 0.25 Units, 0.5 Units, without limitation), a single Unit of insulin (e.g., 1 Unit, without limitation), or multiple Units of Insulin (e.g., 10 Units, 20 Units, 30 Units, 40 Units, 50 Units, 60 Units, 70 Units, 80 Units, 90 Units, or 100 Units, without limitation).

During a given dosing operation, when plunger 408 disrupts optical coupling between detector 404b and light source 402b, detector 404b generates changed output 420. Sensor logic 422 determines which of detectors 404a-404d generated changed output 420 and output location signal 424 which indicates the location of plunger 408. Location signal 424 may be utilized to determine that plunger 408 is located a distance equal to spacing 414 from its starting position for the given dosing operation.

In one or more examples, a totality of detector/light source pairs may be activated during a dosing operation. However, in one or more examples, fewer than a totality of detector/light source pairs depicted by FIG. 4 may be activated for a dosing operation. Activating fewer than a totality of detector/light source pairs may, as a non-limiting example, reduce power utilized by apparatus 300 to perform a dosing operation.

As a non-limiting example, drive controller 312 may, before generating control signals 322 to start a dosing operation, indicate a target ending location of the plunger for the given dosing operation. Sensor logic 422 at location sensor 310 may determine which detector of detectors 404a, 404b, 404c and 404d corresponds to target ending location 426 and activate solely the detector/light source pair for the determined detector. When plunger 408 disrupts optical coupling between the activated detector/light source pair, determined detector generates a changed output that is read by sensor logic 422 and utilized to generate location signal 424.

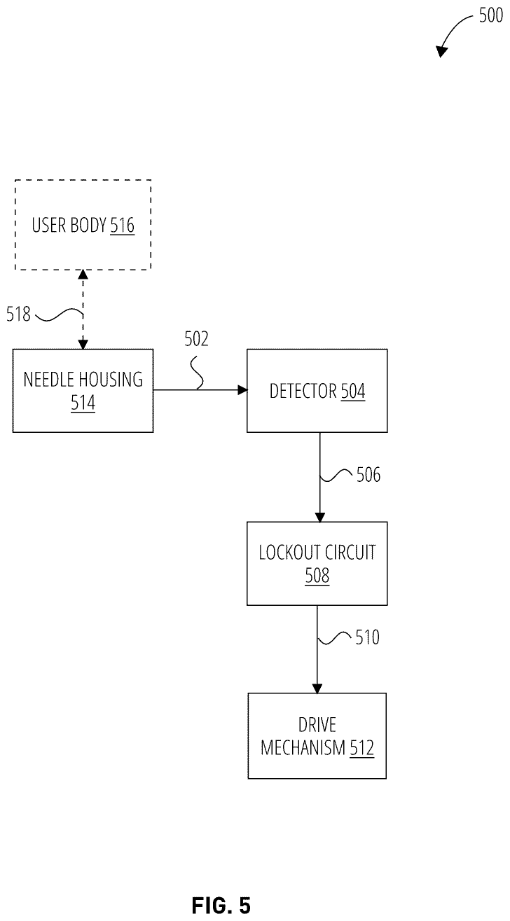

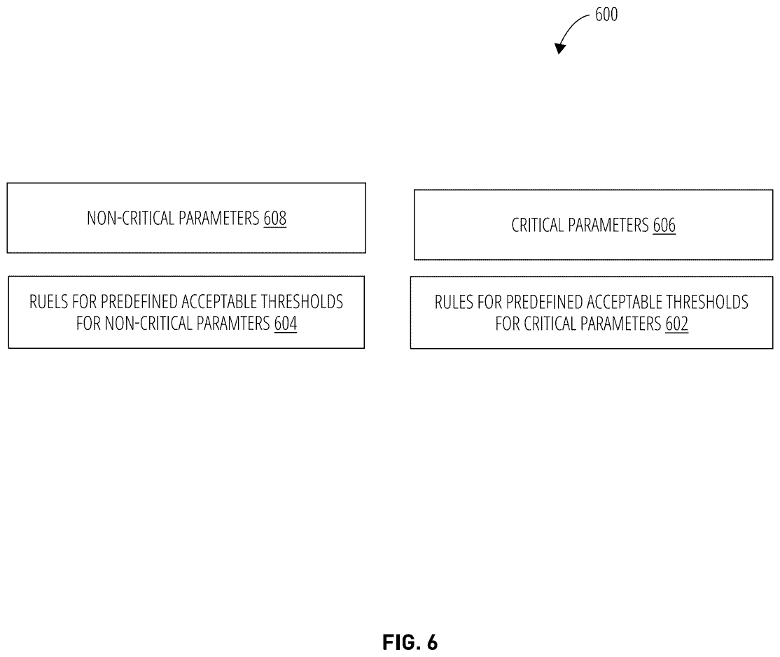

FIG. 5 is a simplified block diagram depicting an apparatus 500 for skin touch detection as a condition to perform a dosing operation at apparatus 300 and dose injection device 102 more generally. As depicted by FIG. 5, apparatus 500 may include a detector 504, a changed output 506, a lockout circuit 508, a drive mechanism 512, a needle housing 514, and a user body 516.

Detector 504 is configured, generally, to detect direct physical contact 518 between needle housing 514 and a user body 516 via an indication of exhibited contact 502. Assuming that a user will place needle housing 514 in direct physical contact 518 with user body 516 (e.g., skin, without limitation) when ready to inject a dose of insulin, detector 504 may be configured to generate changed output 506 responsive to a change in exhibited contact 502 from a first state corresponding to no physical contact between needle housing 514 and user body 516 to a second stage corresponding to direct physical contact between needle housing 514 and user body 516.

In one or more examples, detector 504 may be or include a capacitive sensor, and direct physical contact between needle housing 514 and user body 516 may be exhibited by a change in capacitance of needle housing 514. As a non-limiting example, when needle housing 514 is not in direct physical contact with user body 516 it may exhibit a first capacitance, and when needle housing 514 is in direct physical contact with user body 516 it may exhibit a second capacitance, different than the first capacitance. Detector 504 may detect the change in capacitance from the first capacitance to the second capacitance and generate changed output 506 to indicate a change in state of exhibited contact 502 from no direct physical contact to direct physical contact.

Lockout circuit 508 is configured, generally, to generate a lockout signal at least partially responsive to an output of detector 504. More specifically, lockout circuit 508 is configured to assert lockout signal 510 at least partially responsive to an output of detector 504 indicative of no direct physical contact 518 between needle housing 514 and user body 516, and de-assert lockout signal 510 at least partially responsive to an output of detector 504 indicative of direct physical contact 518 between needle housing 514 and user body 516.

In one or more examples, an enable input of drive mechanism 512 is coupled to receive lockout signal 510. The connection between lockout circuit 508 and enable input of drive mechanism 512 is configured so that when lockout signal 510 is asserted, the enable input is deactivated and when lockout signal 510 is de-asserted, the enable input is activated. When the enable input is activated, drive mechanism 512 may operate and apply force to a plunger (e.g., plunger 324 of FIG. 3, without limitation), and when drive mechanism 512 is deactivated, it may not operate and apply a force to a plunger (e.g., plunger 324, without limitation).

In one or more examples, disclosed dose injection devices may be configured to alternately enable or disable dose injection at least partially responsive to detecting dosing conditions. Dosing conditions may be pre-configured rules at a disclosed dose injection device to be satisfied to deliver a dose via dose injection device 102.

In one or more examples, if dosing conditions are not detected, then the injection mechanism is disabled. If dosing conditions are detected, then the injection mechanism is enabled.

In one or more examples, if dosing conditions are not detected, then a logic circuit of a dose injection device (e.g., logic circuit 314, without limitation) does not generate a dose ready notification. If dosing conditions are detected, then a logic circuit of a dose injection device may generate a dose ready notification. Dosing conditions may include first and second dosing conditions. First dosing conditions are critical dosing conditions, and in one or more examples, logic circuit 314 will request a target dose amount 318 only upon detecting that each and every first dosing condition has been satisfied. Second dosing conditions are, individually, non-critical dosing conditions, and in one or more examples, logic circuit 314 will request a target dose amount 318 only upon detecting that a threshold number of second dosing conditions have been satisfied.

Non-limiting examples of first dosing conditions include meal announcement, dose ready confirmation, dosing alert, state of injection device, last analyte value. Non-limiting examples of first dosing conditions in the environment of a user with diabetes include meal announcement, dose ready confirmation, dosing alert, state of injection device, last glucose value. Examples of such are, if valid current glucose value is above a predetermined threshold corresponding to risk of hypoglycemia (e.g., 50/60/70 mg/dl without limitation), an indication of malfunction of the dose injection device is “false,” an indication of skin-touch detection is “true,” if dose ready confirmation is “true,” etc. Non-limiting examples of second dosing conditions include heart rate, exercise, meal timing, sleep, allergies, menstruation, smoking, temperature, weight, hormones, stress, caffeine and alcohol intake, and medications. Non-limiting examples of second dosing conditions in the environment of a user with diabetes include heart rate, exercise, meal timing, sleep, allergies, menstruation, smoking, temperature, weight, hormones, stress, caffeine and alcohol intake, and medications.

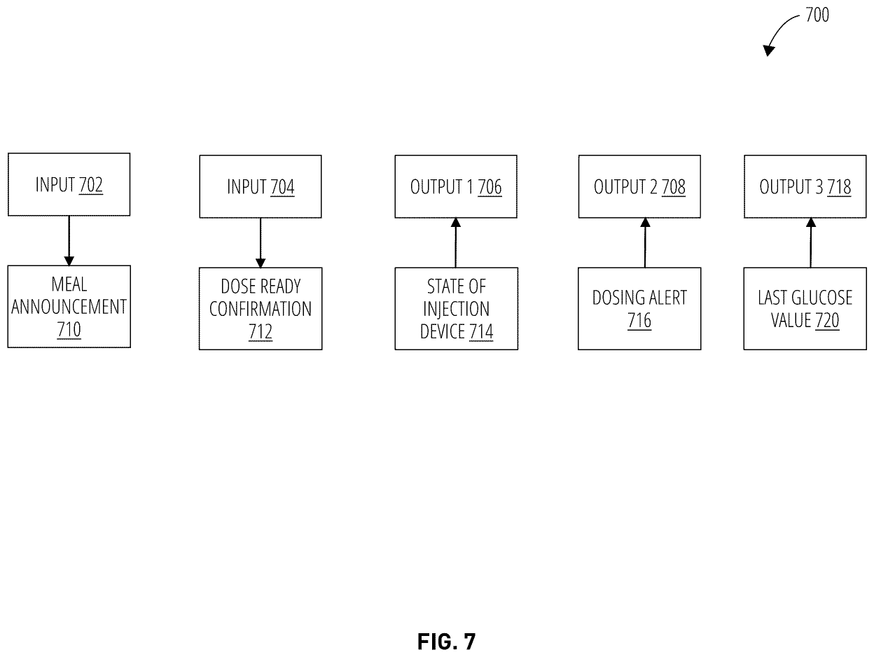

FIG. 7 is a simplified block diagram of a user interface 700 for a user to interact with disclosed dose injection systems and devices, in accordance with one or more examples. In one or more examples, user interface 700 may be provided via any suitable hardware including icons on a touch display, buttons, lights, or dials, without limitation.

User interface 700 may include one or more inputs 702 for a user to provide meal announcements 710 and dose ready confirmation 712 to autonomous dose injection system 802. Input 702 of user interface 700 may be any suitable input for a user to make a meal announcement or delivery confirmation, such as clickable buttons or icons on a touch pad respectively corresponding to meal announcement 710 for small, medium, or large meals. In another example, input 702 may be a dial with an associated analog or digital display that changes when a user rotates the dial. When a user rotates the dial, the associated display may present options to select small, medium, or large meal announcement 710.

User interface 700 may include one or more output for presenting information to a user. In the specific non-limiting example depicted by FIG. 7, user interface 700 includes output 706 to indicate state of injection device 714 and output 708 to indicate dosing alert 716. State of injection device 714 indicated to a user may include that autonomous dose injection system 802 is enabled or disabled. As a non-limiting example, autonomous dose injection system 802 may be disabled when skin touch is not detected and enabled when skin touch is detected, as discussed with reference to FIG. 5, and such state (disabled or enabled) indicated by output 706. As another non-limiting example, state of injection device 714 may be disabled because first dosing conditions or second dosing conditions have not been detected.

Output 708 may present dosing alerts 716 to a user. Dosing alerts notify a user of a time to dose insulin or not a time to dose insulin. Output 718 may present last glucose value 720 to a user. Last glucose value 720 informs a user as discussed above.

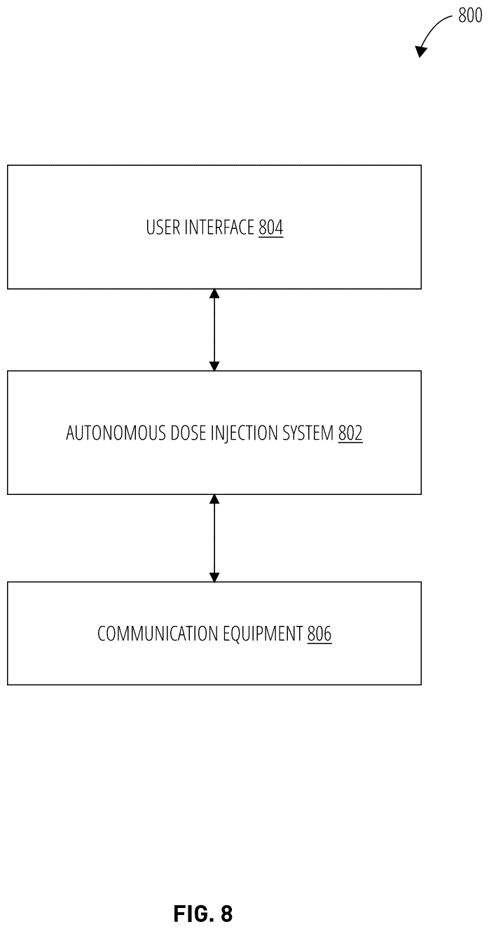

FIG. 8 is a simplified block diagram of a dose injection device 800, in accordance with one or more examples. In the specific non-limiting example depicted by FIG. 8, dose injection device 800 includes autonomous dose injection system 802, user interface 804 and communication equipment 806.

Autonomous dose injection system 802 is a system or apparatus configured, generally, to autonomously inject doses of insulin, such as apparatus 300.

User interface 804 is for a user to interact with dose injection device 800, in accordance with one or more examples.

Communication equipment 806 is communication equipment for dose injection device 800 wireless communicate with analyte sensor 108 and therapy application 106.

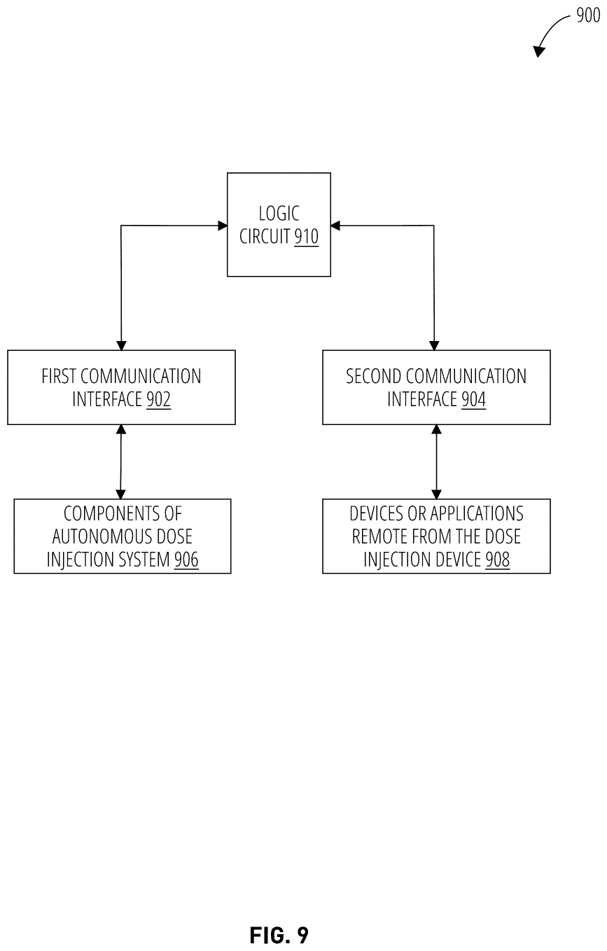

In one or more examples, different communication interfaces may be provided to communicate with components of autonomous dose injection system and devices or applications remote from the dose injection device. FIG. 9 is a simplified block diagram depicting system 900 that utilizes a first communication interface to communicate with components of autonomous dose injection system, and a second communication interface to communicate with devices or applications remote from the dose injection device. In the non-limiting example depicted by FIG. 9, systems 900 includes a first communication interface 902, a second communication interface 904, a components of autonomous dose injection system 906, a devices or applications remote from the dose injection device 908, and a logic circuit 910.

First communication interface 902 is an interface for a communication system (e.g., a communication bus, without limitation) for communicating with components of autonomous dose injection system 906. As a non-limiting example, logic circuit 314 may be configured to use first communication interface 902 to provide target dose amount 318 to drive controller 312 and notifications 330 to user interface 804.

Second communication interface 904 is an interface for a communication system (e.g., a communication bus, without limitation) for communicating with devices or applications remote from the dose injection device 908. As a non-limiting example, logic circuit 910 may utilize second communication interface 904 to communicate with therapy application 106 via communication equipment 806, and more specifically to provide dosing data 118 to therapy application 106.

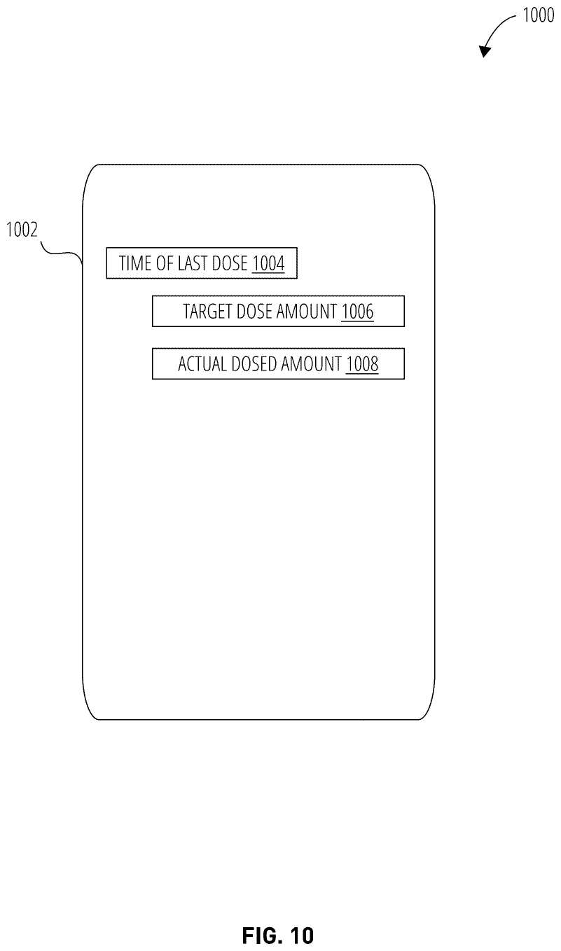

FIG. 10 is a diagram depicting an example display screen 1000 of a mobile device executing a therapy application (e.g., mobile device 104 executing therapy application 106, without limitation). As discussed above, in one or more examples a display screen on a dose injection device 102 does not display values for a target dose amount or a dose amount delivered. In some cases, a user may desire to view such information so in or more examples therapy application 106 may receive target dose amounts and dose amount delivered from dose injection device 102 and therapy application user interface 1002 may display one or more of: a time of last dose in field 1004, target dose amount in field 1006, or dosed amount delivered in field 1008. This information may be updated from time to time at least partially based on dosing data 118 sent by dose injection device 102.

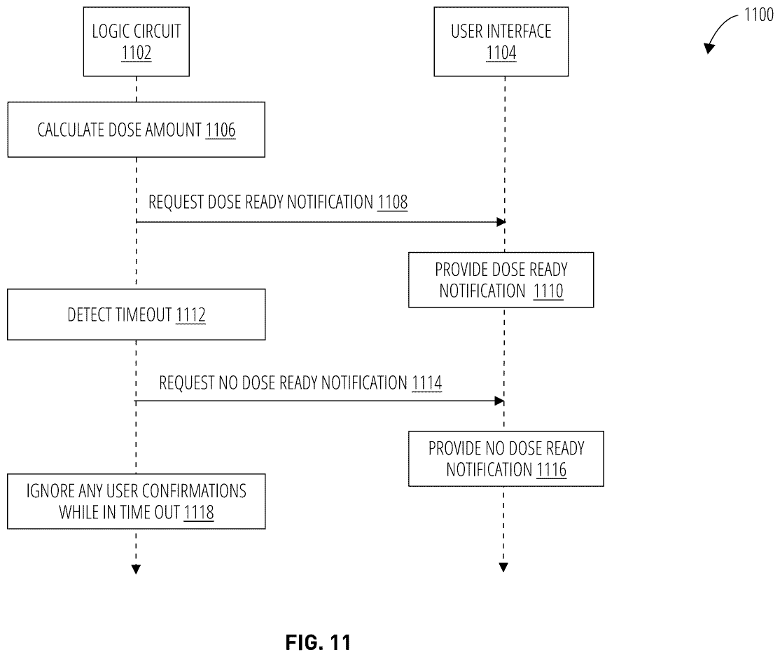

FIG. 11 is a swim lane diagram depicting a process 1100 for controlling dosing, in accordance with one or more examples. Depicted are logic circuit 1102 (e.g., logic circuit 314, without limitation) and user interface 1104 (e.g., user interface 210, without limitation).

At operation 1106, logic circuit 1102 calculates a dose amount, i.e., a target dose amount. At operation 1108, logic circuit 1102 sends user interface 1104 a request to provide a dose ready notification. The dose ready notification is to notify a user that a dose is ready to inject. At operation 1110, user interface 1104 provides the dose ready notification. As discussed, the dose ready notification 212 may be a visual indicator, or a visual indicator and audible indicator recognizable by a user to indicate a dose is of insulin is ready. At operation 1112, logic circuit 1102 detects a time out condition. A time out condition occurs when a predetermined time duration passes from when logic circuit 1102 requested the dose ready notification at operation 1108, without receiving a dosing confirmation from a user. The pre-determined time duration may be set to any desirable time interval. For example, 15 minutes, 30 minutes, 45 minutes, or 1 hour. The time interval may be set based on any of a number of factors, including, but not limited to, a recent analyte value (recent analyte value within normal range so wait a normal amount of time) or a trend in analyte values (increasing analyte values indicate an immediate need to dose insulin so time interval set shorter than a normal time interval). At operation 1114, in response to detecting the time out condition at operation 1112, logic circuit 1102 sends a request to user interface 1104 to not provide the dose ready notification. In response to the request not to provide the dose ready notification, user interface 1104 stops providing the dose ready notification at operation 1116. At operation 1118, logic circuit 1102 optionally ignores any user confirmations received by logic circuit 1102 while in time out or while no request for dosing confirmation is pending.

FIG. 12 is a swim lane type flow diagram depicting a process 1200 for providing a dose of insulin that is a compliment of process 1100. At operation 1208, logic circuit 1102 calculates a target dose amount. At operation 1210, logic circuit 1202 sends user interface 1204 a request to provide a dose ready notification. At operation 1212, user interface 1204 provides the dose ready notification. At operation 1214, user interface 1204 sends a dosing confirmation initiated by a user interacting with user interface 1204. At operation 1216, in response to the dose ready dosing confirmation, logic circuit 1202 requests a dose corresponding to the calculated dose amount to drive controller 1206.

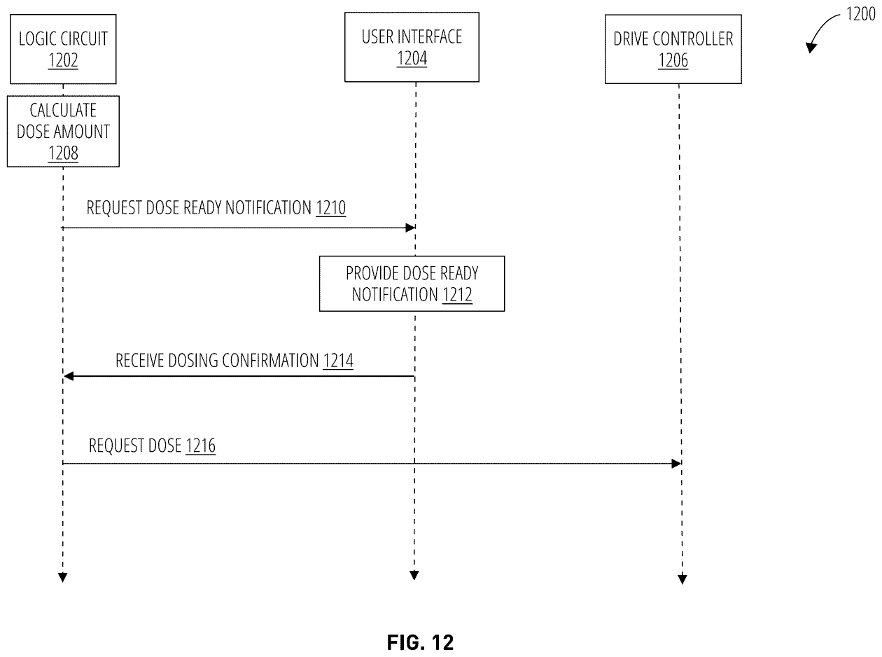

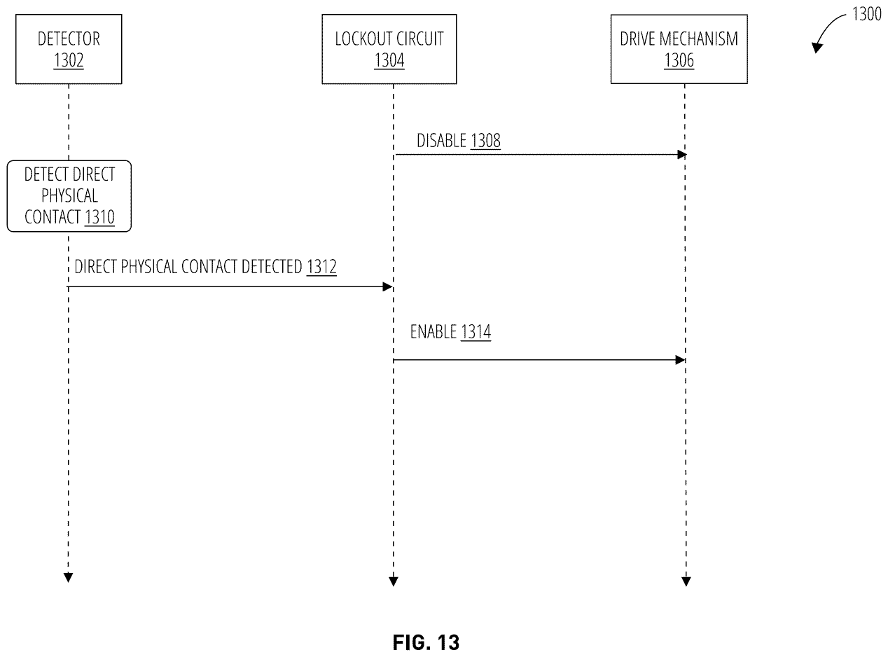

FIG. 13 is a swim lane type flow diagram depicting a process 1300 for injecting a dose of insulin, in accordance with one or more examples. Depicted are detector 1302, lockout circuit 1304 and drive mechanism 1306. At operation 1308, lockout circuit 1304 instructs drive mechanism 1306 to disable (e.g., de-asserts an enablement signal, without limitation). When disabled, drive mechanism 1306 does not respond to requests to deliver a dose. At operation 1310, detector 1302 detects direct physical contact between a need housing (e.g., needle housing 514, without limitation) and a user's body (e.g., user body 516, without limitation). At operation 1312, detector 1302 notifies lockout circuit 1304 that direct physical contact was detected. In response to notification that direct physical contact was detected, in operation 1314, lockout circuit 1304 instructs drive mechanism 1306 to enable (e.g., asserts an enablement signal, without limitation).

FIG. 14 is a swim lane type flow diagram depicting a process 1400 for injecting a dose of insulin, in accordance with one or more examples. Depicted are detector 1402, lockout circuit 1404, and drive mechanism 1406. At operation 1408, lockout circuit 1404 instructs drive mechanism 1406 to disable (e.g., de-asserts an enablement signal, without limitation). When disabled, drive mechanism 1406 does not respond to requests to deliver a dose. At operation 1410, detector 1402 notifies lockout circuit 1404 that first dosing conditions have been detected. In response to notification that first dosing conditions have been detected, in operation 1412, lockout circuit 1404 instructs drive mechanism 1406 to enable (e.g., asserts an enablement signal, without limitation).

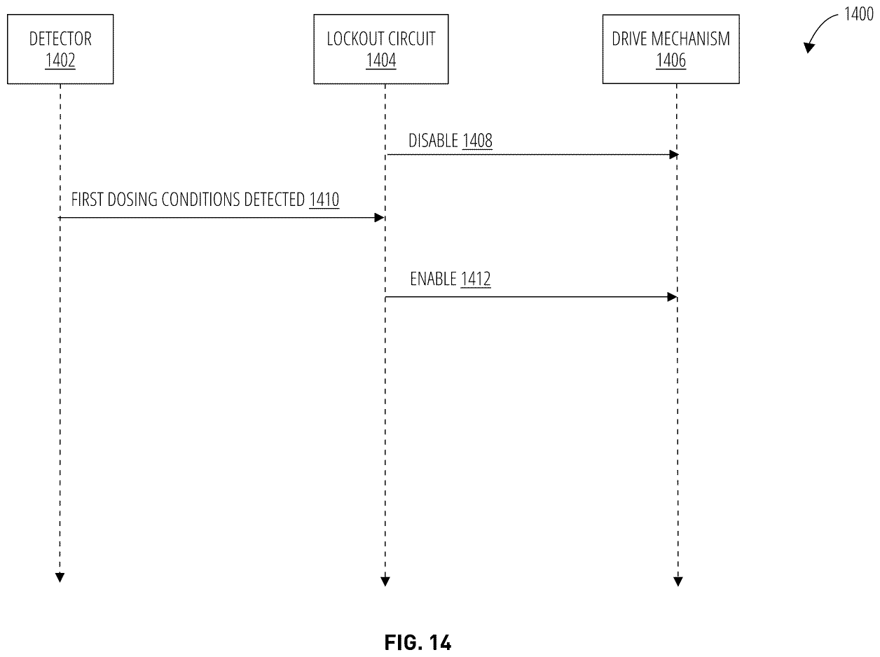

FIG. 15 is a swim lane type flow diagram depicting a process 1500 for injecting a dose of insulin, in accordance with one or more examples. Depicted are detector 1502, lockout circuit 1504, and drive mechanism 1506. At operation 1508, lockout circuit 1504 instructs drive mechanism 1506 to disable (e.g., de-asserts an enablement signal, without limitation). When disabled, drive mechanism 1506 does not respond to requests to deliver a dose. At operation 1510, detector 1502 notifies lockout circuit 1504 that a threshold number of second dosing conditions have been detected. In response to notification that first dosing conditions have been detected, in operation 1512, lockout circuit 1504 instructs drive mechanism 1506 to enable (e.g., asserts an enablement signal, without limitation).

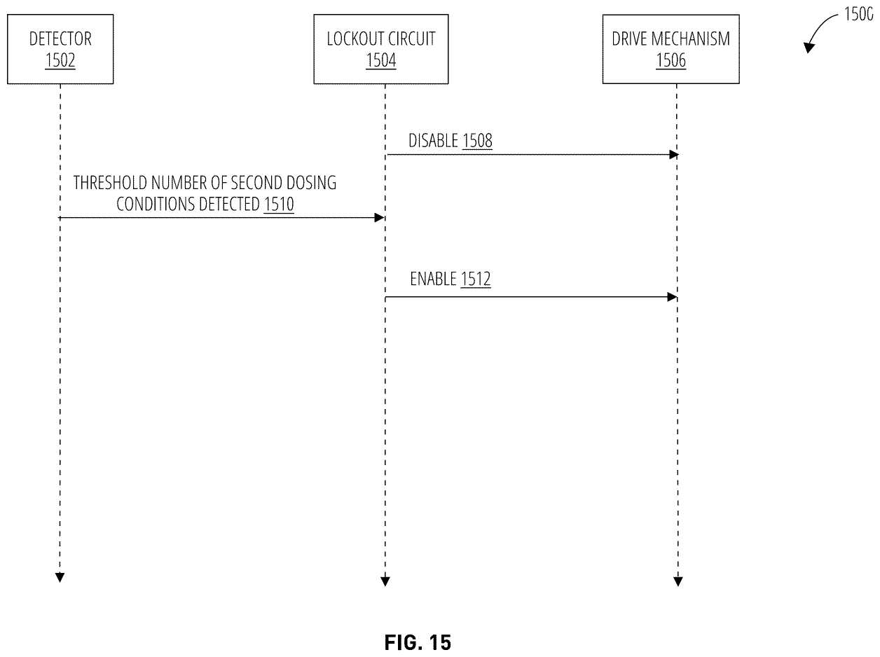

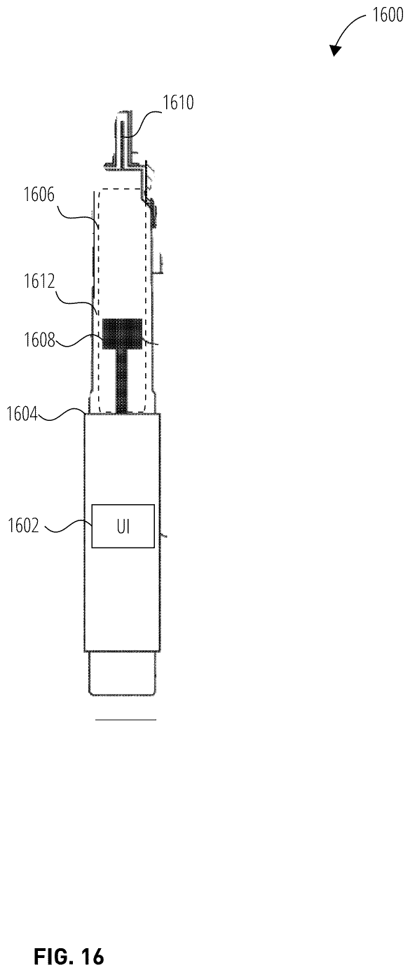

FIG. 16 is a diagram of an injection pen 1600 that is configured as a dose injection device 102 in accordance with one or more examples.

FIG. 16 depicts an injection pen 1600 that includes a housing 1604 and injector 1610. Walls of housing 1604 may be relatively opaque to allow (e.g., filter to, without limitation) only select wavelengths of electromagnetic radiation (e.g., infrared or microwave radiation, without limitation) to transfer there through. Housing 1604 may define an internal volume 1612 (e.g., a reservoir) or a chamber for receiving a disposable pre-filled cartridge 1606 of a drug. The pre-filled cartridge 1606 may include an actuator 1608, for example, a plunger portion in fluid communication with the drug, and configured to communicate a predetermined quantity of the drug to a user. The actuator 1608 may also include a drive mechanism, drive controller, and location sensor, such as discussed with respect to apparatus 300, configurable (e.g., by a logic circuit, without limitation) to dispense variable quantities of a drug. The injector 1610 (e.g., a needle in a needle housing, without limitation) may be configured to penetrate a user's tissue for intramuscular, subcutaneous, and/or intravenous delivery of the drug. In one or more examples, injection pen 1600 may further include communication equipment to send and receive data, for example, with a analyte sensor or therapy application, and a power supply (e.g., a battery, without limitation) to power the actuator 1608, user interface 1602 to enable a user to interact with injection pen 1600 (e.g., display screen 200B or display screen 200C, without limitation), and communication equipment. In one or more examples, injection pen 1600 and dose injection device 102 more generally, may be a durable injection device, usable in a residential building.

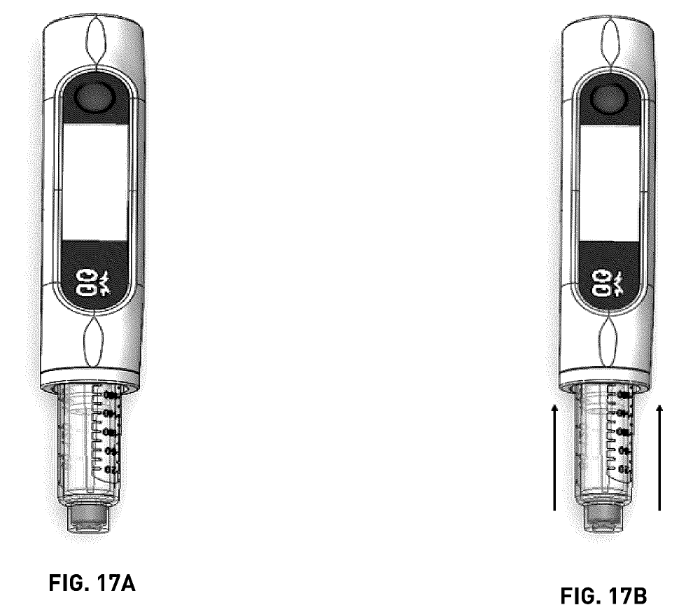

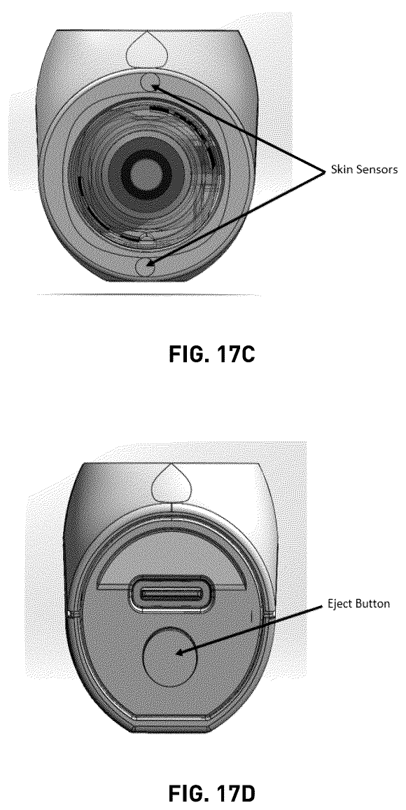

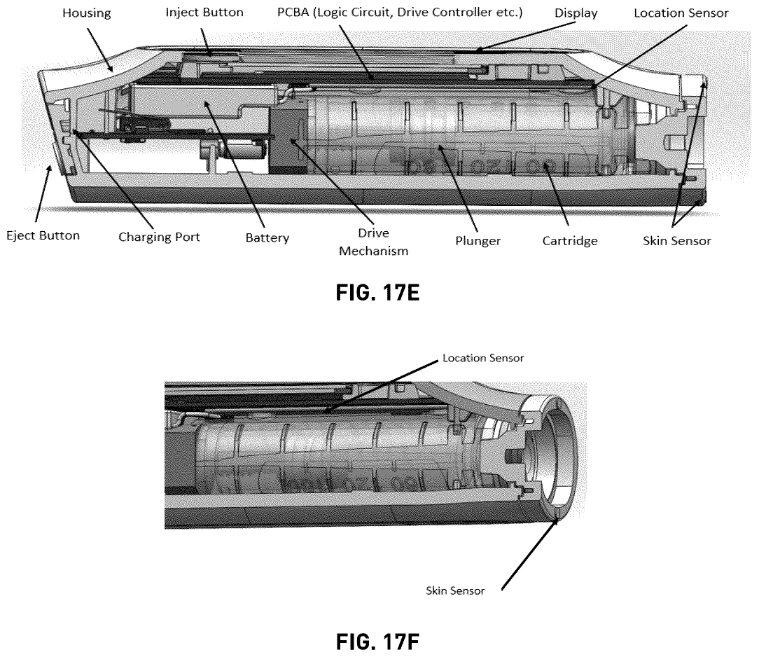

FIGS. 17A-17F illustrate various perspective views of an example dose injection device 108, in accordance with one or more examples.

Examples of autonomous dose injection may be utilized for conditions other than diabetes for example autonomous dose injection may be utilized for blood thinners such as Heparin, palliative care pain medications, growth hormone deficiency (GHD) treatment such as somatotropin, and for fertility treatment such as human chorionic gonadotropin (hCG) or follicle stimulating hormone (FSH). For these treatments, as person having ordinary skill in the art would understand that the first dosing conditions and second dosing conditions discussed herein may be adjusted for the conditions affecting treatment and management.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, without limitation) of the computing system. In one or more examples, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated.

As used in the present disclosure, the term “combination” with reference to a plurality of elements may include a combination of all the elements or any of sub-combinations of some of the elements. For example, the phrase “A, B, C, D, or combinations thereof” may refer to any one of A, B, C, or D; the combination of each of A, B, C, and D; and any sub-combination of A, B, C, or D such as A, B, and C; A, B, and D; A, C, and D; B, C, and D; A and B; A and C; A and D; B and C; B and D; or C and D.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” without limitation).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, without limitation” or “one or more of A, B, and C, without limitation.” is used, in general such a construction is intended to include A alone, B alone, C alone, A and B together, A and C together, B and C together, or A, B, and C together, without limitation.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.” While the present disclosure has been described herein with respect to certain illustrated examples, those of ordinary skill in the art will recognize and appreciate that the present invention is not so limited. Rather, many additions, deletions, and modifications to the illustrated and described examples may be made without departing from the scope of the invention as hereinafter claimed along with their legal equivalents. In addition, features from one example may be combined with features of another example while still being encompassed within the scope of the invention as contemplated by the inventor.

1-38. (canceled)

39. A dose injection device, comprising:
a chamber configured to receive a cartridge;
a drive mechanism configured to apply force to a plunger of a received cartridge, wherein the plunger is arranged to move within the received cartridge responsive to force applied by the drive mechanism and thereby push fluid in the received cartridge toward an interface for dispensing fluid;
a sensor configured to generate a location signal indicative of a plunger location;
a logic circuit configured to calculate a target amount of insulin to deliver to a user; and
a drive controller configured to generate control signals for the drive mechanism, wherein the control signals correspond to the target amount of insulin calculated by the logic circuit.

40. The dose injection device of claim 39, wherein the drive controller is configured to:
generate a first control signal to instruct the drive mechanism to apply force to the plunger,
wherein the first control signal is at least partially responsive to user-action at the dose injection device.

41. The dose injection device of claim 39, wherein the drive controller is configured to:
generate a second control signal to instruct the drive mechanism to stop applying force to the plunger, and
wherein the drive controller is configured to generate the second control signal at least partially responsive to the location signal indicative of a plunger location.

42. The dose injection device of claim 41, wherein the drive controller is configured to:
detect that a location of the plunger indicated by the location signal indicative of plunger location is a predetermined plunger location associated with the target amount of insulin.

43. The dose injection device of claim 39, wherein the drive controller is configured to:
determine a plunger location indicative of a delivered amount of insulin substantially equal to the target amount of insulin calculated by the logic circuit.

44. The dose injection device of claim 39, wherein the logic circuit is configured to request the calculated target amount of insulin at least partially responsive to detecting first conditions, wherein first dosing conditions include possession, by the dose injection device, of one or more of: value for recent blood analyte level, a time since last dose, a value for blood analyte level recent to last dose, and values for therapy settings.

45. The dose injection device of claim 44, wherein the logic circuit is configured to request the calculated target amount of insulin at least partially responsive to detecting a threshold number of second dosing conditions, wherein second dosing conditions include possession of: a value for a blood analyte level trend.

46. The dose injection device of claim 39, further comprising:
a detector configured to detect direct-physical contact between needle housing and user-body; and
a lockout circuit configured to alternately enable or disable the drive mechanism at least partially responsive to an output of the detector.

47. The dose injection device of claim 39, further comprising:
communication equipment configured to wirelessly interface with a glucose monitor and a therapy management application executing at a mobile device, and
wherein the logic circuit is configured to calculate the target amount of insulin to deliver to the user at least partially based on analyte data and therapy parameters received via the communication equipment.

48. The dose injection device of claim 39, further comprising:
communication equipment configured to wirelessly interface with a therapy management application executing at a mobile device; and
wherein one or both of a delivery amount of insulin or the target amount of insulin is accessible to a user solely via the therapy management application.

49. The dose injection device of claim 39, further comprising:
communication equipment configured to wirelessly interface with a therapy management application executing at a mobile device; and
wherein one or both of a delivery amount of insulin or the target amount of insulin is not accessible to a user via the dose injection device.

50. The dose injection device of claim 39, further comprising:
a first interface for communicating with components of the dose injection device; and
a second interface for communicating with devices or applications remote from the dose injection device,
wherein one or both of a delivery amount of insulin or the target amount of insulin is available solely via the second interface.

51. The dose injection device of claim 39, further comprising:
a memory configured to store a time since last dose; and
a lockout circuit configured to alternately enable or disable the drive mechanism at least partially responsive to a value of the time since last dose, wherein the lockout circuit to disable the drive mechanism at least partially responsive to detecting that the value of the time since last dose is outside a predetermined range to dose, and wherein the lockout circuit to enable the drive mechanism at least partially responsive to detecting that the value of the time since last dose is within a predetermined range to dose.

52. The dose injection device of claim 39, further comprising:
a lockout circuit configured to alternately enable or disable the drive mechanism; and
an interface configured to alternately indicate that the drive mechanism is disabled or that the drive mechanism is enabled.

53. The dose injection device of claim 39,
wherein the dose injection device is configured to not provide the user with an indication of the calculated target amount of insulin for non-zero calculated target amounts of insulin.

54. A drug therapy management system, comprising:
a analyte sensor;
a therapy application;
a dose injection device configured to:
calculate a target amount of insulin to deliver to a user's body at least partially responsive to therapy parameters received from the therapy application and blood analyte values received from the analyte sensor;
provide a dose ready notification associated with the calculated target amount of insulin; and
deliver a dose of insulin that corresponds to the calculated target amount of insulin at least partially responsive to receipt of a dosing confirmation.

55. The drug therapy management system of claim 54, wherein the dose injection device to deliver the dose of insulin is at least partially responsive to detecting receipt of the dosing confirmation while providing the dose ready notification associated with the calculated target amount of insulin.

56. A method, comprising:
calculating a target amount of insulin to deliver to a user;
providing a dose ready notification associated with the calculated target amount of insulin;
receiving a dosing confirmation in response to the dose ready notification; and
requesting delivery of the target amount of insulin at least partially responsive to receiving the dosing confirmation.

57. The method of claim 56, wherein the dose ready notification does not disclose the target amount of insulin to deliver and does not disclose a delivered amount of insulin.
